(19) United States
(12) Patent Application Publication
(10) Pub. No.: US 2016/0168198 A1
(43) Pub. Date: Jun. 16, 2016

(54) SIGNAL SEQUENCE FOR PROTEIN EXPRESSION IN PICHIA PASTORIS

(71) Applicant: Biocon Limited, Bangalore (IN)

(72) Inventors: Nagaraj Govindappa, Bangalore (IN); Suma Sreenivas, Bangalore (IN); Sankar Periyasamy, Tirupur (IN); Srividya Divakar, Devasandra, K.R. Puram (IN)

(21) Appl. No.: 14/904,035

(22) PCT Filed: Jun. 27, 2014
(86) PCT No.: PCT/IB2014/062682

§ 371 Date: Jan. 8, 2016

(30) Foreign Application Priority Data
Jul. 1, 2013 (IN) 2905/CHE/2013

(51) Int. Cl. C07K 7/08; C07K 14/415; C12N 15/81

(52) U.S. Cl. CPC C07K 7/08; C12N 15/815; C07K 14/415; C07K 2319/02; C07K 2319/50

(57) ABSTRACT

The present invention relates to a signal sequence from a unique Pichia pastoris protein. Further the invention discloses use of signal sequence for the expression of heterologous protein in Pichia pastoris.

### RELATED APPLICATION

This application claims benefit of Indian Provisional Application No. 2905/CHE/2013 filed on Jul. 1, 2013; all of which are hereby incorporated by reference in their entirety.

### FIELD OF INVENTION

The present invention relates to a signal sequence of 18 amino acids from a unique Pichia pastoris protein. Further the invention discloses use of signal sequence for the expression of heterologous protein in Pichia pastoris.

### BACKGROUND AND PRIOR ART OF THE INVENTION

Signal sequences are N-terminal extensions of nascent polypeptide chains that mediate protein targeting to the membrane of the endoplasmic reticulum (ER). They are on average 16 to 30 amino acid residues in length comprising a characteristic tripartite structure: (1) a hydrophilic, usually positively charged n-region, (2) a central hydrophobic h-region of 5-15 residues and (3) a c-region with the cleavage site for signal peptidase (SPase). The consensus cleavage site consists of amino acids with short side chains at the −1 and no charged amino acid residues at the −3 position. Besides these common features, signal sequences can be quite different in a. a sequence length and mode of function.

Signal peptides control the entry of virtually all proteins to the secretory pathway, both in eukaryotes and prokaryotes. They comprise the N-terminal part of the amino acid (aa) chain and are cleaved off while the protein is translocated through the membrane. The presence of N-terminal signal sequences leads to strong stimulation and expression of recombinant proteins. The proteins secreted from eukaryotic cells require an N-terminal signal peptide.

A signal sequence means a sequence included at the beginning of the coding sequence of a protein to be expressed on the surface of a cell. This sequence encodes a signal peptide, N-terminal to the mature polypeptide, that directs the host cell to translocate the polypeptide.

Currently the secretory expression in Pichia pastoris makes use of the pre-pro mat alpha factor sequence from Saccharomyces cerevisiae and the dibasic amino acid cleavage site (KR) at the end of signal sequence. This efficiently processed by the Kex2 protease (Kex2p) in the host resulting in the secretion of mature proteins to the medium with desired N-terminal amino acid. However, the proteins which have one or more internal dibasic amino acids such as KR and RR in the coding region cannot be expressed using this signal sequence as the protein gets fragmented.

Hence, there exists a need in the art to produce a full length heterologous protein in the medium having dibasic amino acid cleavage site (KR and RR). The inventors of the present invention have found that the identified signal sequence will be very useful to express the heterologous protein having internal Kex2p cleavage sites using Kex2p disrupted Pichia pastoris strain.

### STATEMENT OF THE INVENTION

The present disclosure relates to a signal sequence of 18 amino acids from a unique Pichia pastoris protein. Further the invention discloses use of signal sequence for the expression of heterologous protein in Pichia pastoris.

In an embodiment of the present invention said signal sequence of 18 amino acids is identified and isolated from Pichia pastoris.

In another embodiment of the present disclosure, analysis of extracellular protein profile of all the inactivated strains revealed that the ˜30 KDa proteins being secreting into the medium from the Kex2p disrupted Pichia pastoris.

In still another embodiment of the present disclosure the protein of a single band within a gel after electrophoretic separation of secreted proteins was subjected to N-terminal sequencing by Edman degradation method and the amino acids sequence derived are, “DQTFGVLLIRSG”.

In another embodiment of the present disclosure, the 18 amino acid signal sequence was not present at the N-terminus of the secreted protein and this protein is secreting only upon Kex2p disrupted Pichia pastoris.

In an embodiment of the present disclosure, 18 amino acid at the N-terminus from P. pastoris were identified in by the following process;
a) cloning the 30 KDa protein under the control of strong AOX1 promoter inframe with this novel signal sequence and secreted protein in wild type P. pastoris strain;
b) expressing various constructs and transforming into P. pastoris strain to evaluate and characterize the signal sequence.
c) inducing the resulting strains with methanol using the shake flask experiments.

The results proved that the 18 amino acid signal peptide indeed has a potential to secrete the heterologous proteins efficiently into the medium and that this signal sequence is cleaved of independently without the use of Kex2p cleavage sites.

In still another embodiment of the present disclosure, the signal sequence is more useful in expressing the recombinant proteins with internal di-basic amino acids such as KR and RR Kex2p cleavage sites.

### BRIEF DESCRIPTION OF THE ACCOMPANYING FIGURES

The features of the present disclosure will become more fully apparent from the following description taken in conjunction with the accompanying drawings. Understanding that the drawings depict only several embodiments in accordance with the disclosure and are therefore not to be considered limiting of its scope, the disclosure will be described with additional specificity and detail through use of the accompanying drawings:

FIG. 1: Secretion of 30 KDa DDDK protein in the gene inactivated host.

FIG. 2A: The construct were develop to evaluate the novel signal sequence properties of the 18 amino acids of N-terminus of DDDK protein under the regulatory control of AOX1 promoter.

FIG. 2B (i): Analysis of DDDK protein expression in P. pastoris wild type and PMT1 gene knock out host

FIG. 3: The amino acid sequence of the 30 KDa DDDK protein identified by blasting against the P. pastoris database.

FIG. 4A: The 30 KDa DDDK protein secreted into the medium when expressed the protein under the regulatory control of AOX1promoter in wild type P. pastoris; and

FIG. 4B: Comparison of 30 KDa DDDK protein secretion when inserted Kex2p cleavage site after 18 amino acid signal sequence.

FIG. 5: The Western blot analysis of the 30 KDa DDDK protein to identify whether any protein accumulates intracellularly due to non-processing of signal sequence.

FIG. 6A: The porcine Carboxypeptidase B expressed using DDDK signal sequence has secreted into the medium as efficiently as one secreted with Matα signal sequence.

FIG. 6B: The Erythrina Trypsin Inhibitor (ETI) expressed using DDDK signal sequence has secreted into the medium is comparable to the one secreted with Matα signal sequence

FIG. 7: The primers used for genetic manipulation of the strains developed.

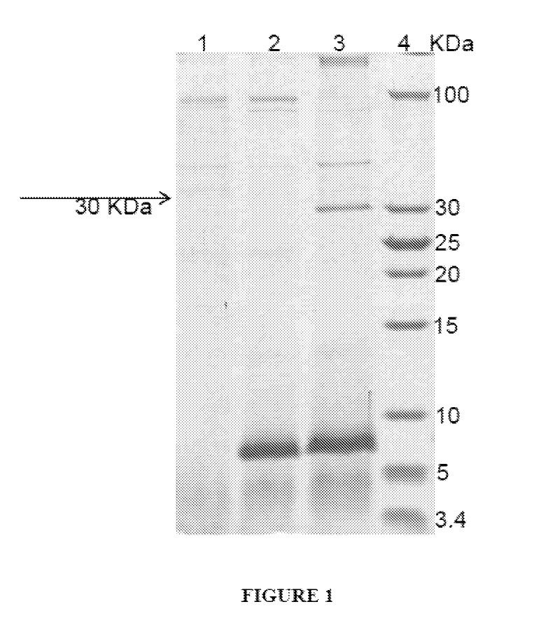

### DETAILED DESCRIPTION OF THE INVENTION

The present invention relates to a signal sequence of 18 amino acids from a unique Pichia pastoris protein. Further the invention discloses use of signal sequence for the expression of heterologous protein in Pichia pastoris.

In an embodiment of the present invention said amino acid is identified and isolated from Pichia pastoris.

In another embodiment of the present disclosure, analysis of extracellular protein profile of all the inactivated strains revealed that the ˜30 KDa protein (SEQ ID NO: 4) being secreting into the medium from the Kex2p disrupted Pichia pastoris.

In still another embodiment of the present disclosure, the protein band representing the ˜30 KDa protein was subjected to N-terminal sequencing by Edman degradation method and the amino acids sequence derived is DQTFGVLLIRSG (SEQ ID NO: 3).

In another embodiment of the present disclosure the 18 amino acid at the N-terminus were not part of the secreted protein and this protein is secreting into the medium from the Kex2p disrupted Pichia pastoris.

In an embodiment of the present disclosure; 18 amino acid at the N-terminus from P.pastoris were identified in by the following process;

a) First cloned this 30 KDa protein under the control of strong AOX1 promoter inframe with this novel signal sequence and secreted protein in wild type P.pastoris strain.

b) Various expressions construct and transformed into P.pastoris strain to evaluate and characterize the signal sequence.

c) the resulting strains were induced with methanol using the shake flask experiments and results proved that the 18 amino acid signal peptide indeed has a potential to secrete the heterologous proteins efficiently into the medium and this signal sequence is cleaved of independently without the use of Kex2p cleavage sites.

In still another embodiment of the present disclosure; signal sequence is more useful in expressing the recombinant proteins with internal di-basic amino acids such as KR and RR Kex2p cleavage sites.

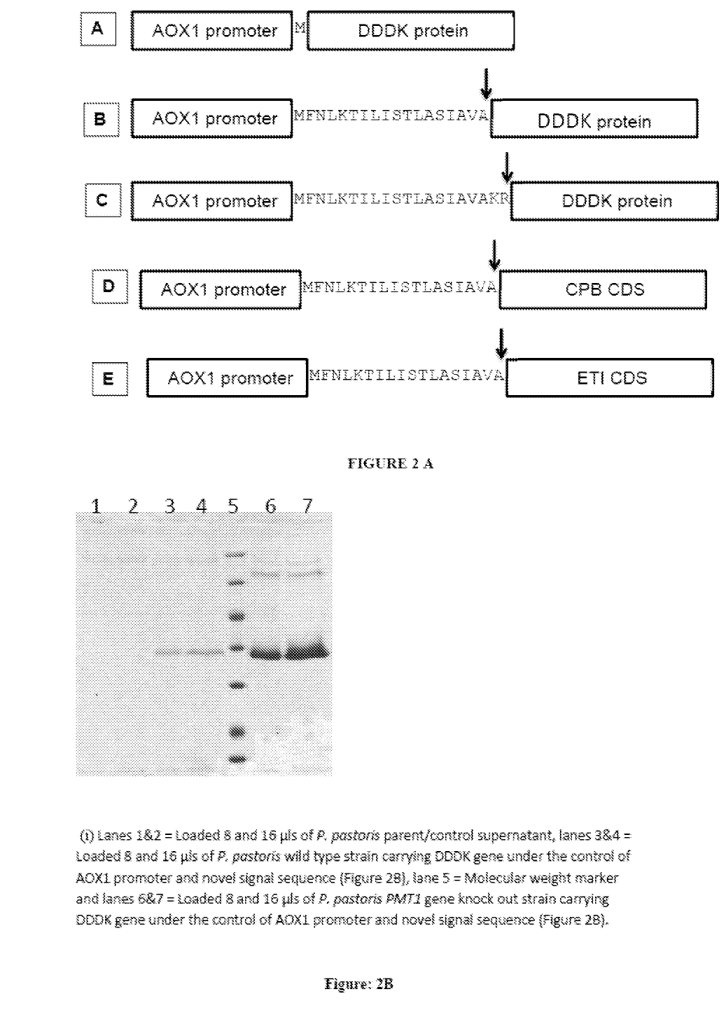

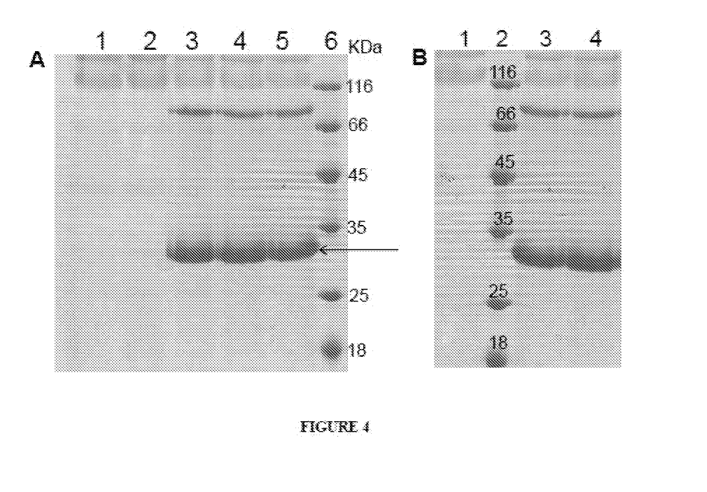

In an embodiment of the present invention; the signal sequence of DDDK protein can be used for the expression of recombinant protein. The DDDK protein along with its signal sequence under AOX1 promoter (FIG. 2B) and successfully secreted the protein in wild type P. pastoris strain (FIG. 4).

According to the most significant aspects of the present disclosure; a) the 18 amino acid signal sequence is able to secrete the proteins into the medium, b) the secretion of protein is independent of the Kex2p cleavage after signal sequence and c) the strong promoter is able secrete same protein in wild type P. pastoris also.

In another embodiment of the present disclosure; P. pastoris strain BICC 9450 [9] and its derivative BICC 9682 (his⁻) hosts were used for the current studies and modified pMBL 208 expression vector which provides the promoter and terminator sequences of the P. pastoris AOX1 gene.

In still another embodiment of the present disclosure; The S. cerevisiae Matα signal sequence was replaced with “MFNLKTILISTLASIAVA” signal sequence for secretion of proteins into the medium. The E. coli strain DHSα was used for routine cloning and propagation of plasmids.

In yet another embodiment of the present disclosure; Yeast extract-peptone-dextrose (YPD) used in medium containing 10 g/liter yeast extract, 20 g/liter peptone and 20 g/liter dextrose was used for routine growth and subculturing of Pichia strains. YNBD medium used for selection contained 13.4 g/liter yeast nitrogen base (YNB) without amino acids and 20 g/liter dextrose (D). Luria broth/agar was used for culturing E. coli.

As used herein, “heterologous” means not naturally contiguous. For example, a yeast secretion signal peptide sequence and a human polypeptide sequence are heterologous because the two sequences are not naturally contiguous.

In another embodiment of the present disclosure, the most preferred host cells are methylotrophic yeasts. Strains of a methylotrophic yeast which can be modified using the present disclosure include, but are not limited to yeast strains capable of growing on methanol, such as yeasts of the genera. Pichia, Candida, Hansenula, or Torulopsis. Preferred methylotrophic yeasts are of the genus Pichia. Methylotrophic yeast strains which can be modified using the present methods also include those methylotrophic yeast strains which have been engineered to express one or more heterologous proteins of interest.

In another embodiment of the present disclosure, the term “promoter” is used in the normal sense of the art, e.g. an RNA polymerase binding site.

The ten “promoter” is used in the normal sense of the art, e.g. an RNA polymerase binding site.

The term “vector” includes expression vectors, replicable vectors, transformation vectors and shuttle vectors, including vector combinations thereof.

The term “expression vector” means a construct capable of in-vivo or in-vitro expression, Preferably the expression vector is incorporated in the genome of the organism.

The term “incorporated” preferably covers stable incorporation into the genome.

As used herein the term “expression” refers to a process by which a polypeptide is produced based on the nucleic acid sequence of a gene. The process includes both transcription and translation.

The terms “polypeptide,” “peptide” and “protein” are used interchangeably herein to refer to a polymer of amino acid residues of any length.

The term “polypeptide of interest” refers to any polypeptide that may be expressed in a host cell as disclosed herein, preferably in a strain of Pichia pastoris as disclosed herein. The polypeptide of interest may be purified from the culture medium said host cell is or has been cultivated in during expression of said polypeptide of interest. The polypeptide of interest may for example be be an enzyme, a hormone, a ligand for a receptor or an antibody.

The term “construct”, as used herein, refers to any recombinant polynucleotide molecule. Examples of constructs may be a plasmid, a cosmid, a virus, an autonomously replicating polynucleotide molecule, a phage, or a linear or circular single-stranded or double-stranded DNA or RNA polynucleotide molecule, derived from any source, capable of genomic integration or autonomous replication, comprising a polynucleotide molecule where one or more polynucleotide molecule(s) has been linked in a functionally operative manner, i.e., operably linked

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH).

As used herein, the term “signal sequence” refers to a sequence of amino acid residues bound at the amino terminus of a nascent protein during protein translation, which when recognized by the signal recognition particle results in the transport of the nascent protein to the organelle of destination. During protein translation, a specific protein is being produced through the action of mRNA and ribosome. A signal sequence comprised of amino acid residues may be produced eventually and bound as part of the growing protein.

The signal sequence is like a “flag” bound at the amino terminus of the emerging protein. This “flag” signals the transport mechanism of the cell to prompt them as to where the emerging protein should go. This “flag” is recognized by signal recognition particle (SRP) located freely in the cytoplasm or attached to the ribosome.

The SRP interacts with the signal sequence, which leads to the transient arrest of translation. At this time, the protein (not yet fully formed) is transported to the destination organelle, e.g. endoplasmic reticulum especially when the protein is for secretion. Upon docking to the membrane of the endoplasmic reticulum the SRP detaches and this leads to the continuation of protein translation, and eventually to the maturation of protein in which the signal sequence is cleaved by the signal peptidase.

The invention is further illustrated by the following examples. The following examples are provided for illustrative purposes only and are not intended to limit the scope of the invention.

### EXPERIMENTAL EXAMPLES

### Example 1

Construction of the Expression Constructs for Secretory Expression in P. pastoris

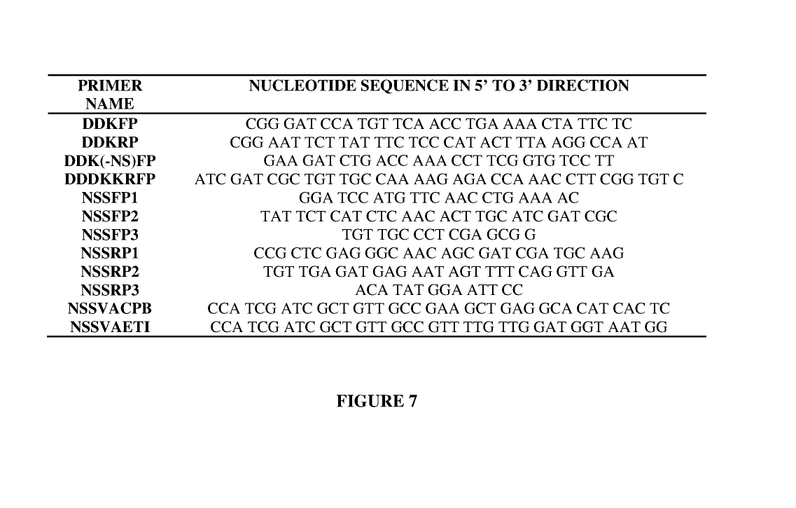

The gene corresponding to the DDDK protein (SEQ ID NO: 4) was amplified along with signal sequence using primers DDKFP (SEQ ID NO: 6) and DDKRP (SEQ ID NO: 7) (FIG. 7) and P. pastoris genomic DNA template. The 870 bps PCR product was cloned into pTZ57R vector, sequence verified. The gene excised using BamHI and EcoRI sites and subcloned into pMBLNSS208 vector in BamHI and EcoRI restriction sites to obtain DDK/pMBLNSS208 vector which replaces the Matα signal sequence. The DDDK gene is under the regulatory control of AOX1 promoter (SEQ ID NO: 19) (FIG. 2B). The DDDK protein ORF without the signal sequence (SEQ ID NO: 5) was amplified and cloned similarly to have a control for the above (FIG. 2A). The forward primer used was DDK(-SS)FP (SEQ ID NO: 8) in place of DDKFP. These two constructs were used to study whether the strong promoter can lead to secretion of this protein in the wild type Pichia. The constructs Fig.2C is similar FIG. 2B but with the Kex2p site (KR) at the end of signal sequence. This was developed to evaluate the level of secretion with and without Kex2p cleavage site.

### Example 2

Synthesis of Signal Sequence and Development of CPB and ETI Clones

The signal sequence was synthesized using the primers NSSFP1 (SEQ ID NO: 10), NSSFP2 (SEQ ID NO: 11), NSSFP3 (SEQ ID NO: 12), NSSRP1 (SEQ ID NO: 13), NSSRP2 (SEQ ID NO: 14) and NSSRP3 (SEQ ID NO: 15) (FIG. 7). The primers were phosphorylated by treating with T4 polynucleotide kinase, annealed and ligated. The full length signal sequence was amplified from the ligated template using NSSFP1 (SEQ ID NO: 10) and NSSRP1 (SEQ ID NO: 13). The PCR product was cloned into pTZ57R vector and sequence verified. The signal sequence was cloned into pMBL208 vector to replace the Matα signal sequence.

The CPB codsing sequence (SEQ ID NO: 20) and the ETI coding sequence (SEQ ID NO: 24) were amplified using the forward primers NSSVACPB (SEQ ID NO: 16) and NSSVAETI (SEQ ID NO: 17) and pPIC9K reverse primer (SEQ ID NO: 18) from the plasmid CPB/pPIC9K and ETI/pPIC9K which were expressed previously in our laboratory. The forward primer has the ClaI restriction site. The PCR product was digested with ClaI and EcoRI and cloned into the DDK/pMBLNSS208 vector in the identical sites to yield the constructs FIG. 2D and FIG. 2E. These constructs will not have amino acids ‘KR’, the Kex2p cleavage sites at the N-terminus of mature coding region.

### Example 3

Transformation of Pichia pastoris

The plasmids constructs were digested with SacI and transformed into P. pastoris (his⁻) strain. Transformation was carried out by electroporation of freshly prepared competent cells in 0.2 cm cuvettes. The pulses were delivered by Gene Pulser (BioRad) at 1500 V, 25 μF, and 200 Ω. The electroporated cells were allowed to recover for 1 hour in 1M Sorbitol at 30° C. and then spread on to YNBD agar plates. The resulting transformants were selected on YPD plates, containing 0.5 mg/ml of G418 concentrations to identify multicopy clones. Several clones that were viable at 0.5 mg/ml concentration were tested in small scale induction experiments. The proteins secreted to the extracellular medium were analyzed on 10% SDS-PAGE.

### Example 4

Induction of the Proteins Expression in P. pastoris

The clones were grown on 10 ml YNB medium (1.34% yeast nitrogen base w/o amino acids and 2% dextrose) overnight. It was sub cultured into 50 ml BMGY (1% yeast extract, 2% peptone, 1.34% YNB, 100 mM potassium phosphate at pH 6.0 and 1% glycerol) with 0.5 OD₆₀₀. This culture was incubated at 30° C. for 48 h with shaking at 220 rpm. The cells were harvested by centrifugation at 5000 rpm for 5 min at room temperature and resuspended in induction medium (1% yeast extract, 2% peptone, 1.34% YNB and 100 mM potassium phosphate at pH 6.0). Methanol (2%) was added to the culture medium to give a final concentration of 0.5% methanol as an inducer. Samples were collected every day and analyzed by SDS-PAGE.

A construct shown in FIG. 2B was transformed into P. pastoris wild type and PMT1 gene inactivated P. pastoris (US 2012/20309935) and the clones were shortlisted as described in example 3 and 4. The DDDK protein secreted to the medium was analyzed on 12% SDS PAGE.

Summary: It is very clear from the gel picture that the DDDK protein secretion is enhanced when expressed in PMT1 gene knock out host as it is evident from lanes 6 and 7 as compared to corresponding lanes 3 and 4.

### Example 5

Western Blotting

Western blotting was carried out as per the method described by Towbin with few modifications. The proteins samples were separated on 12% SDS-PAGE and then transferred to the Nitrocellulose sheet, 0.2 μM (Bio-Rad Laboratories, USA) as described above. After transfer, the membrane was soaked in 1% BSA in Tris NaCl buffer (20 mM Tris pH 7.2, 150 mM glycine) and incubated overnight to block the non-specific binding. The membrane was washed with wash buffer (20 mM Tris pH 7.2, 150 mM glycine, 0.05% Tween-20) thoroughly. The membrane was probed with an anti-DDK monoclonal antibody (OriGene Technologies, Inc, Rockville, Md. 20850) at 1:100 dilution with Tris NaCl buffer containing 0.1% BSA. The DDK protein was identified with a polyclonal Sheep anti-mouse IgG-HRP conjugate (product code: NA931, GE Health care, UK Limited) using TMB substrate.

### Example 6

Amino Acid Analysis and Identification of Signal Sequence

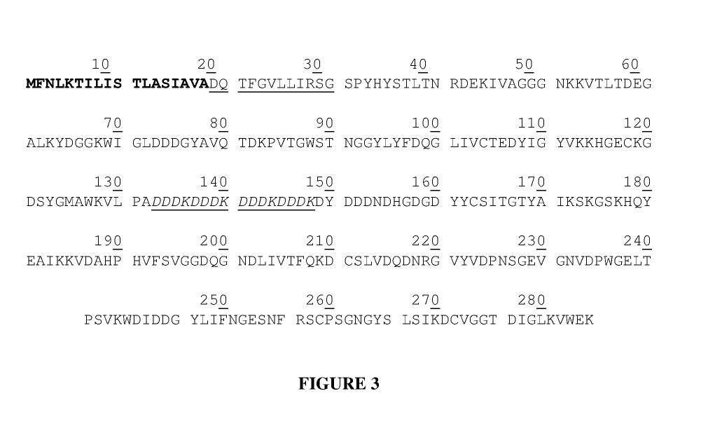

The 30 KDa protein secreted in the PMT1 gene inactivated P. pastoris strain was immobilized onto Sequi-blot-PVDF membrane and submitted for N-terminal a a sequencing at ProSeq. Inc. USA. The first 12 aa were found to be “DQTFGVLLIRSG” (SEQ ID NO: 3). This sequence was blasted against the NCBI database Blastp program to find out the probable homologous protein. It resulted in the identification of a 289 aa containing hypothetical protein in the P. pastoris GS 115 genome database (SEQ ID NO: 4). There are 18 aa at the N-terminal of the protein (SEQ ID NO: 1) were acting as a signal sequence (FIG. 3) and were cleaved off during the secretion of protein to the mature protein into the medium. The theoretical mass of this protein is found to be 31.66 KDa with its pI of 4.44 when calculated using ExPASY protein parameter tool. It is highly acidic protein with four ‘DDDK’ aa repeats at a stretch in the primary a sequence.

### Example 7

Secretion of DDDK Protein Under AOX1 Promoter in Wild Type P. pastoris

In order to evaluate whether this protein will come out if fused with strong promoter AOX1 (SEQ ID NO: 19). The 870 bps complete DDDK ORF which includes the coding sequence of 18 aa signal peptide was cloned under the regulatory control of AOX1 promoter (FIG. 2B). The construct was transformed into the P. pastoris wild type strain. The clones obtained were screened for the secretory production of the DDDK protein and the good secretory expression of DDDK protein in the wild type P. pastoris strain (FIG. 4). This indicates that the promoter of the native protein is indeed very weak and may not be able sufficiently produce the sufficient amount of protein to secrete into the medium, although it has a potential signal sequence at the N-terminus. The results shown in FIG. 4B indicates that the signal sequence can secrete the protein independent of Kex2p cleavage site as evident from the similar band intensity in lane 3 and 4.

### Example 8

Identification of Production of DDDK Protein Using Antibody in Wild Type

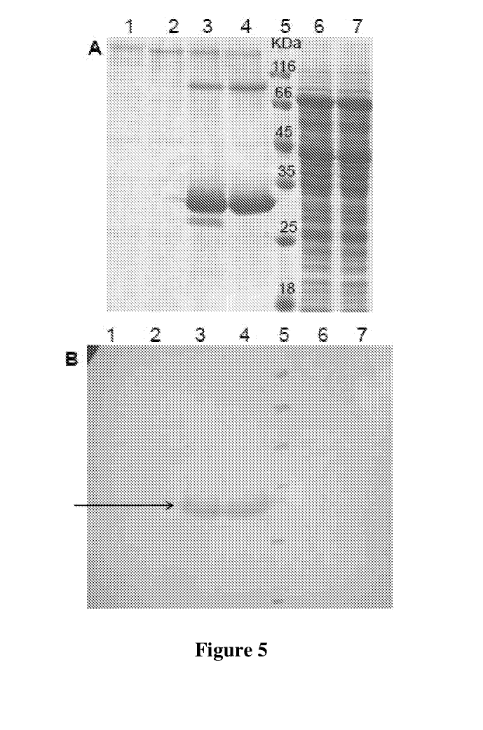

Western blot experiment using a monoclonal antibody that bind to proteins which have DDDK motif was carried out. The protein was not detected in the supernatants of Pichia wild type strain and Pichia strain carrying DDDK protein under the regulatory control of AOX1 promoter. It was seen in any band corresponds to DDK protein in the wild type Pichia strain (lane 1, FIG. 5) indicating that the protein is being secreted only from the Kex2p disrupted Pichia pastoris. It was also noticed that there was no band observed in the intracellular fraction of Pichia proteins indicating that all the protein expressed has successfully secreted to the medium with use of this novel DDDK signal sequence.

### Example 9

Secretion of CPB and ETI Using DDK Protein Signal Sequence

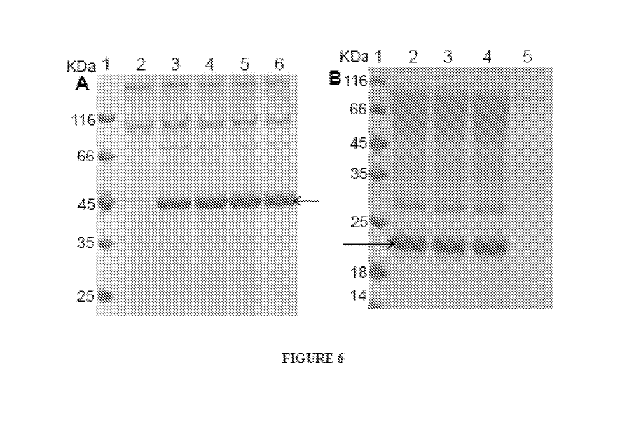

To show whether the signal sequence functions if we use for secreting heterologous proteins, we have developed the constructs having CPB and ETI coding sequences (SEQ ID NOs: 20 and 24) fused in frame with this signal sequence (SEQ ID NO: 2) (FIGS. 2A-D and E) without the Kex2p cleavage sites. The resulting nucleic acid sequences are represented by SEQ ID NOs: 22 and 26. The constructs were transformed into the P. pastoris wild type strain. The clones obtained were screened for the secretory production of these proteins. We found the good secretory expression of CPB (SEQ ID NO: 21) and ETI (SEQ ID NO: 25) proteins which are equivalent the proteins produced using mat-α signal sequence (FIG. 6).

The present invention comprises the following embodiments:
A. The signal peptide of the DDDK protein, wherein the signal peptide has the amino acid sequence MFNLKTILISTLASIAVA (SEQ ID NO: 1).
B. The signal peptide according to embodiment A, wherein said signal peptide can be isolated from a Pichia pastoris strain.
C. The signal peptide according to embodiment A, wherein the amino acid sequence can be obtained by using a nucleic acid molecule comprising or consisting of codon, wherein any existing codon for a given amino acid can be present in said nucleic acid molecule.
D. A nucleic acid molecule comprising the nucleotide sequence of SEQ ID NO: 2.
E. A nucleic acid molecule comprising a nucleotide sequence encoding the amino acid sequence according to SEQ ID NO: 1, wherein said nucleic sequence has an identity of at least 80% to the nucleotide sequence of SEQ ID NO: 2.
F. A method of Kex2p-independent protein secretion using the signal peptide of SEQ ID NO: 1 the nucleotide sequence of SEQ ID NO: 2 and/or the nucleotide sequence having at least 80% identity to the nucleotide sequence of SEQ ID NO: 2.
G. A Pichia pastoris host cell comprising a PMT1 gene knock out and a nucleic acid molecule comprising the nucleotide sequence of SEQ ID NO: 2 or a nucleotide sequence having at least 80% identity to the nucleotide sequence of SEQ ID NO: 2.
H. Use of the signal peptide of the DDDK protein for heterologous protein expression in P. pastoris.
I. Porcine carboxypeptidase B expressed in P. pastoris using a nucleic acid molecule wherein nucleotide sequence encoding the porcine carboxypeptidase B is fused in frame the nucleic acid sequence encoding the P. pastoris DDDK signal peptide.
J. Erythrina Trypsin Inhibitor expressed in P. pastoris using a nucleic acid molecule wherein nucleotide sequence encoding the Erythrina Trypsin Inhibitor B is fused in frame the nucleic acid sequence encoding the P. pastoris DDDK signal peptide.

1. A heterologous nucleic acid molecule comprising a nucleotide sequence encoding the amino acid sequence according to SEQ ID NO: 1.

2. The heterologous nucleic acid molecule according to claim 1, wherein the nucleotide sequence encoding the amino acid sequence according to SEQ ID NO: 1 consists of the nucleotide sequence according to SEQ ID NO: 2.

3. The heterologous nucleic acid molecule according to claim 1, wherein the nucleotide sequence encoding the amino acid sequence according to SEQ ID NO: 1 consists of a nucleotide sequence having a similarity of at least 80% to the nucleotide sequence according to SEQ ID NO: 2.

4. The heterologous nucleic acid molecule according to claim 1, wherein the nucleic acid molecule comprises a nucleotide sequence encoding a polypeptide of interest, the nucleotide sequence encoding said polypeptide of interest being present contiguously (in frame) at and to the 3′ end of nucleotide sequence encoding the amino acid sequence according to SEQ ID NO: 1 such that the nucleotide sequence encoding the amino acid sequence according to SEQ ID NO: 1 and the nucleotide sequence encoding said polypeptide provide a continuous open reading frame for a heterologous polypeptide comprising the amino acid sequence according to SEQ ID NO: 1 at its N-terminal end.

5. The heterologous nucleic acid molecule according to claim 1, wherein said nucleic acid molecule further comprise a nucleotide sequence representing a promoter, said nucleotide sequence representing a promoter being present at a position within the heterologous nucleic acid molecule such that the promoter controls expression of the nucleotide sequence encoding the amino acid sequence according to SEQ ID NO: 1 or the expression of the nucleotide sequence encoding the heterologous polypeptide.

6. The heterologous nucleic acid molecule according to claim 5, wherein the promoter is an inducible promoter, preferably the AOX1 promoter.

7. A heterologous polypeptide comprising the amino acid sequence according to SEQ ID NO: 1.

8. A host cell comprising a heterologous nucleic acid molecule as defined in claim 1.

9. Use of a polypeptide consisting of the amino acid sequence according to SEQ ID NO: 1 and/or a nucleic acid sequence encoding the amino acid sequence according to SEQ ID NO: 1 for having a polypeptide of interest that is expressed in Pichia pastoris being secreted into the culture medium.

10. The use according to claim 9, wherein the polypeptide of the amino acid sequence according to SEQ ID NO: 1 constitutes the N-terminal end of a heterologous polypeptide, said heterologous polypeptide further comprising the polypeptide of interest.

11. A method for producing a polypeptide, said method comprising the steps of:
a) transforming a host cell with a nucleic acid molecule, wherein the nucleotide sequence encoding a polypeptide of interest is contiguously fused to a nucleotide sequence encoding the DDDK signal peptide of SEQ ID NO: 1 such that a continuous open reading frame is provided which encodes a heterologous fusion protein consisting of the P. pastoris DDDK signal peptide and the polypeptide of interest; and
b) inducing expression of the heterologous fusion protein.

12. The method according to claim 11, further comprising isolation of the polypeptide of interest which was expressed as heterologous polypeptide the host cell from the culture medium said host cell was cultivated in during expression.

13. The method according to claim 11, wherein the host cell is selected from the group consisting of yeasts of the genera Pichia, Candida, Hanensula and Torulopsis.

14. The method according to any one of claims 11, wherein the host cell is a host cell having its Kex2p gene disrupted.

15. The method according to any one of claims 11, wherein the expression of the heterologous fusion protein is under control of an inducible promoter.

16. The method according to claim 15, wherein the inducible promoter is the AOX 1 promoter.

17. The method according to claim 11, wherein the nucleotide sequence encoding the polypeptide of interest is a nucleotide sequence selected from the group of nucleotide sequences encoding a carboxypeptidase B and nucleotide sequences encoding a erythrina trypsin inhibitor.

18. A host cell expressing a heterologous polypeptide as defined in claim 7.


SEQUENCE LISTING (27 sequences)

SEQ ID NO: 1 - protein, 18 aa, Pichia pastoris
MFNLKTILISTLASIAVA

SEQ ID NO: 2 - DNA, 54 bp, Pichia pastoris
atgttcaacctgaaaactattctcatctcaacacttgcatcgatcgctgttgcc

SEQ ID NO: 3 - protein, 12 aa, Pichia pastoris
DQTFGVLLIRSG

SEQ ID NO: 4 - protein, 289 aa, Pichia pastoris
MFNLKTILISTLASIAVADQTFGVLLIRSGSPYHYSTLTNRDEKIVAGGGNKKVTLTDEGALKYDGGKWIGLDDDGYAVQTDKPVTGWSTNGGYLYFDQGLIVCTEDYIGYVKKHGECKGDSYGMAWKVLPADDDKDDDKDDDKDDDKDYDDDNDHGDGDYYCSITGTYAIKSKGSKHQYEAIKKVDAHPHVFSVGGDQGNDLIVTFQKDCSLVDQDNRGYVYDPNSGEVGNVDPWGELTPSVKWDIDDGYLIFNGESNFRSCPSGNGYSLSIKDCVGGTDIGLKVWEK

SEQ ID NO: 5 - protein, 271 aa, Pichia pastoris
DQTFGVLLIRSGSPYHYSTLTNRDEKIVAGGGNKKVTLTDEGALKYDGGKWIGLDDDGYAVQTDKPVTGWSTNGGYLYFDQGLIVCTEDYIGYVKKHGECKGDSYGMAWKVLPADDDKDDDKDDDKDDDKDYDDDNDHGDGDYYCSITGTYAIKSKGSKHQYEAIKKVDAHPHVFSVGGDQGNDLIVTFQKDCSLVDQDNRGYVYDPNSGEVGNVDPWGELTPSVKWDIDDGYLIFNGESNFRSCPSGNGYSLSIKDCVGGTDIGLKVWEK

SEQ ID NO: 6 - DNA, 32 bp, Artificial
  forward primer for amplification of P. pastoris DDDK open reading frame.
cgggatccatgttcaacctgaaaactattctc

SEQ ID NO: 7 - DNA, 35 bp, Artificial
  reverse primer for amplification of P. pastoris DDDK open reading frame.
cggaattcttatttctcccatactttaaggccaat

SEQ ID NO: 8 - DNA, 29 bp, Artificial
  forward primer for amplification of a truncated DDDK open reading frame.
gaagatctgaccaaaccttcggtgtcctt

SEQ ID NO: 9 - DNA, 40 bp, Artificial
  reverse primer for amplifying the P. pastoris DDDK gene.
atcgatcgctgttgccaaaagagaccaaaccttcggtgtc

SEQ ID NO: 10 - DNA, 23 bp, Artificial
  forward primer for amplifying the P. pastoris DDDK signal sequence.
ggatccatgttcaacctgaaaac

SEQ ID NO: 11 - DNA, 30 bp, Artificial
  forward primer for amplifying a truncated P. pastoris DDDK signal seqeunce.
tattctcatctcaacacttgcatcgatcgc

SEQ ID NO: 12 - DNA, 16 bp, Artificial
  forward primer for amplifying a truncated P. pastoris DDDK signal seqeunce.
tgttgccctcgagcgg

SEQ ID NO: 13 - DNA, 30 bp, Artificial
  reverse primer for amplifying the P. pastoris DDDK signal sequence
ccgctcgagggcaacagcgatcgatgcaag

SEQ ID NO: 14 - DNA, 29 bp, Artificial
  reverse primer for amplifying a truncated P. pastoris DDDK signal sequence.
tgttgagatgagaatagttttcaggttga

SEQ ID NO: 15 - DNA, 14 bp, Artificial
  reverse primer for amplifying a truncated P. pastoris DDDK signal sequence.
acatatggaattcc

SEQ ID NO: 16 - DNA, 38 bp, Artificial
  forward primer for amplifying the CPB coding seqeunce.
ccatcgatcgctgttgccgaagctgaggcacatcactc

SEQ ID NO: 17 - DNA, 38 bp, Artificial
  forward primer for amplifying the ETI coding sequence.
ccatcgatcgctgttgccgttttgttggatggtaatgg

SEQ ID NO: 18 - DNA, 20 bp, Artificial
  reverse primer
tgcccaacttgaactgagga

SEQ ID NO: 19 - DNA, 946 bp, Pichia pastoris
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatccgat

SEQ ID NO: 20 - DNA, 1221 bp, Sus sp.
atggaagctgaggcacatcactctggtgaacattttgaaggagagaaagttttcagagttaatgttgaagatgagaacgacatttctttgcttcatgagttggcctccactagacaaattgacttctggaagccagattcagtcactcaaatcaaaccacactctaccgttgactttagagttaaggctgaggatattttggctgttgaggactttctggaacagaatgagttgcaatacgaggttttgatcaacaatttgagatctgttcttgaggctcaatttgactctagagtcagaacaactggacattcctacgagaagtataataactgggagactattgaagcctggactaaacaagtcacttcagagaatcctgatctgatttctagaactgccattggtaccacatttctgggtaacaatatctacttgctgaaagttggtaagcctggacctaataagccagctatctttatggattgtggatttcatgctagagagtggatttctcatgcattctgtcaatggtttgttagagaggctgttttgacttatggatacgagtctcacatgactgagtttcttaacaagttggacttctacgttttgccagttttgaacattgatggttacatctacacttggactaagaatagaatgtggagaaagactagatccactaatgctggtacaacttgtattggtactgatccaaacagaaactttgacgctggatggtgtactaccggtgcctcaactgatccatgtgacgagacttactgtggttctgctgccgagtctgagaaagagactaaagctttggctgactttatcagaaataacttgtcatccattaaagcttacttgactattcattcttattctcaaatgattttgtatccatactcctacgattacaagttgcctgagaacaatgcagagttgaataacctggctaaggctgccgtcaaagagcttgccactttgtacggtactaagtacacttacggtccaggtgctactaccatttacccagctgcaggtggatctgatgactgggcatacgatcaaggtatcaagtactcttttacctttgagttgagagacaaaggtagatacggatttatcttgcctgagtctcaaattcaagctacttgtgaagagactatgttggctattaagtacgtcactaactacgttttgggacacttgtaa

SEQ ID NO: 21 - protein, 406 aa, Sus sp.
MEAEAHHSGEHFEGEKVFRVNVEDENDISLLHELASTRQIDFWKPDSVTQIKPHSTVDFRVKAEDILAVEDFLEQNELQYEVLINNLRSVLEAQFDSRVRTTGHSYEKYNNWETIEAWTKQVTSENPDLISRTAIGTTFLGNNIYLLKVGKPGPNKPAIFMDCGFHAREWISHAFCQWFVREAVLTYGYESHMTEFLNKLDFYVLPVLNIDGYIYTWTKNRMWRKTRSTNAGTTCIGTDPNRNFDAGWCTTGASTDPCDETYCGSAAESEKETKALADFIRNNLSSIKAYLTIHSYSQMILYPYSYDYKLPENNAELNNLAKAAVKELATLYGTKYTYGPGATTIYPAAGGSDDWAYDQGIKYSFTFELRDKGRYGFILPESQIQATCEETMLAIKYVTNYVLGHL

SEQ ID NO: 22 - DNA, 1272 bp, Artificial
  coding sequence for porcine CPB comprising the P. pastoris DDDK signal sequence.
atgttcaacctgaaaactattctcatctcaacacttgcatcgatcgctgttgccgaagctgaggcacatcactctggtgaacattttgaaggagagaaagttttcagagttaatgttgaagatgagaacgacatttctttgcttcatgagttggcctccactagacaaattgacttctggaagccagattcagtcactcaaatcaaaccacactctaccgttgactttagagttaaggctgaggatattttggctgttgaggactttctggaacagaatgagttgcaatacgaggttttgatcaacaatttgagatctgttcttgaggctcaatttgactctagagtcagaacaactggacattcctacgagaagtataataactgggagactattgaagcctggactaaacaagtcacttcagagaatcctgatctgatttctagaactgccattggtaccacatttctgggtaacaatatctacttgctgaaagttggtaagcctggacctaataagccagctatctttatggattgtggatttcatgctagagagtggatttctcatgcattctgtcaatggtttgttagagaggctgttttgacttatggatacgagtctcacatgactgagtttcttaacaagttggacttctacgttttgccagttttgaacattgatggttacatctacacttggactaagaatagaatgtggagaaagactagatccactaatgctggtacaacttgtattggtactgatccaaacagaaactttgacgctggatggtgtactaccggtgcctcaactgatccatgtgacgagacttactgtggttctgctgccgagtctgagaaagagactaaagctttggctgactttatcagaaataacttgtcatccattaaagcttacttgactattcattcttattctcaaatgattttgtatccatactcctacgattacaagttgcctgagaacaatgcagagttgaataacctggctaaggctgccgtcaaagagcttgccactttgtacggtactaagtacacttacggtccaggtgctactaccatttacccagctgcaggtggatctgatgactgggcatacgatcaaggtatcaagtactcttttacctttgagttgagagacaaaggtagatacggatttatcttgcctgagtctcaaattcaagctacttgtgaagagactatgttggctattaagtacgtcactaactacgttttgggacacttgtaa

SEQ ID NO: 23 - protein, 423 aa, Artificial
  porcine carboxypeptidase B comprising the P. pastoris DDDK signal sequence.
MFNLKTILISTLASIAVAEAEAHHSGEHFEGEKVFRVNVEDENDISLLHELASTRQIDFWKPDSVTQIKPHSTVDFRVKAEDILAVEDFLEQNELQYEVLINNLRSVLEAQFDSRVRTTGHSYEKYNNWETIEAWTKQVTSENPDLISRTAIGTTFLGNNIYLLKVGKPGPNKPAIFMDCGFHAREWISHAFCQWFVREAVLTYGYESHMTEFLNKLDFYVLPVLNIDGYIYTWTKNRMWRKTRSTNAGTTCIGTDPNRNFDAGWCTTGASTDPCDETYCGSAAESEKETKALADFIRNNLSSIKAYLTIHSYSQMILYPYSYDYKLPENNAELNNLAKAAVKELATLYGTKYTYGPGATTIYPAAGGSDDWAYDQGIKYSFTFELRDKGRYGFILPESQIQATCEETMLAIKYVTNYVLGHL

SEQ ID NO: 24 - DNA, 522 bp, Erythrina sp.
atggttttgttggatggtaatggtgaagttgttcaaaatggtggtacttattatttgttgccacaagtttgggctcaaggtggtggtgttcaattggctaagactggtgaagaaacttgtccattgactgttgttcaatctccaaatgaattgtctgatggtaagccaattagaattgaatctagattgagatctgcttttattccagatgatgataaggttagaattggttttgcttatgctccaaagtgtgctccatctccatggtggactgttgttgaagatgaacaagaaggtttgtctgttaagttgtctgaagatgaatctactcaatttgattatccatttaagtttgaacaagtttctgatcaattgcattcttataagttgttgtattgtgaaggtaagcatgaaaagtgtgcttctattggtattaatagagatcaaaagggttatagaagattggttgttactgaagattatccattgactgttgttttgaagaaggatgaatcttcttaa

SEQ ID NO: 25 - protein, 173 aa, Erythrina sp.
MVLLDGNGEVVQNGGTYYLLPQVWAQGGGVQLAKTGEETCPLTVVQSPNELSDGKPIRIESRLRSAFIPDDDKVRIGFAYAPKCAPSPWWTVVEDEQEGLSVKLSEDESTQFDYPFKFEQVSDQLHSYKLLYCEGKHEKCASIGINRDQKGYRRLVVTEDYPLTVVLKKDESS

SEQ ID NO: 26 - DNA, 573 bp, Artificial
  coding sequence for Erythrina Trypsin Inhibito comprising the P. pastoris DDDK signal sequence.
atgttcaacctgaaaactattctcatctcaacacttgcatcgatcgctgttgccgttttgttggatggtaatggtgaagttgttcaaaatggtggtacttattatttgttgccacaagtttgggctcaaggtggtggtgttcaattggctaagactggtgaagaaacttgtccattgactgttgttcaatctccaaatgaattgtctgatggtaagccaattagaattgaatctagattgagatctgcttttattccagatgatgataaggttagaattggttttgcttatgctccaaagtgtgctccatctccatggtggactgttgttgaagatgaacaagaaggtttgtctgttaagttgtctgaagatgaatctactcaatttgattatccatttaagtttgaacaagtttctgatcaattgcattcttataagttgttgtattgtgaaggtaagcatgaaaagtgtgcttctattggtattaatagagatcaaaagggttatagaagattggttgttactgaagattatccattgactgttgttttgaagaaggatgaatcttcttaa

SEQ ID NO: 27 - protein, 537 aa, Artificial
  Erythrina Trypsin Inhibitor comprising the P. pastoris DDDK signal sequence.
MFNLKTILISTLASIAVAGTTTTGTTGGATGGTAATGGTGAAGTTGTTCAAAATGGTGGTACTTATTATTTGTTGCCACAAGTTTGGGCTCAAGGTGGTGGTGTTCAATTGGCTAAGACTGGTGAAGAAACTTGTCCATTGACTGTTGTTCAATCTCCAAATGAATTGTCTGATGGTAAGCCAATTAGAATTGAATCTAGATTGAGATCTGCTTTTATTCCAGATGATGATAAGGTTAGAATTGGTTTTGCTTATGCTCCAAAGTGTGCTCCATCTCCATGGTGGACTGTTGTTGAAGATGAACAAGAAGGTTTGTCTGTTAAGTTGTCTGAAGATGAATCTACTCAATTTGATTATCCATTTAAGTTTGAACAAGTTTCTGATCAATTGCATTCTTATAAGTTGTTGTATTGTGAAGGTAAGCATGAAAAGTGTGCTTCTATTGGTATTAATAGAGATCAAAAGGGTTATAGAAGATTGGTTGTTACTGAAGATTATCCATTGACTGTTGTTTTGAAGAAGGATGAATCTTCTTAA